(19) United States
(12) Patent Application Publication
(10) Pub. No.: US 2026/0056106 A1
(43) Pub. Date: Feb. 26, 2026

(54) Method for estimating a viscosity curve of a polymeric material

(71) Applicant: AT & S Austria Technologie & Systemtechnik AG, Leoben (AT)

(72) Inventor: Qi Tao, Leoben (AT)

(21) Appl. No.: 19/101,823

(22) PCT Filed: Aug. 31, 2022
(86) PCT No.: PCT/EP2022/074217

§ 371 Date: Feb. 6, 2025

(51) Int. Cl. G01N 11/00; G01N 33/44

(52) U.S. Cl. CPC G01N 11/00; G01N 33/442; G01N 2011/002

(57) ABSTRACT

The invention relates to a method for estimating a viscosity curve of a polymeric material, the method comprising: acquiring viscosity values of the polymeric material at different temperatures (T) under different heating rates (β); determining at least one viscosity curve of the polymeric material depending on the measured viscosities for each heating rate (β); splitting the viscosity curves into at least one determined melting curve and at least one determined curing curve per determined viscosity curve; determining at least one fitted melting curve per determined melting curve; determining at least one fitted curing curve per determined curing curve; and estimating the viscosity curve of the polymeric material by combining the fitted melting curve and the fitted curing curve.

### FIELD OF THE INVENTION

The invention relates to a method for estimating a viscosity curve of a polymeric material.

### BACKGROUND OF THE INVENTION

In general, a polymeric material may comprise one or more polymers, or a mixture of one or more polymers and one or more fillers. Polymeric materials, such as prepregs, solder masks, glues, ABFs etc., are important base materials for electronic devices and/or components of electronic devices, e.g., for PCBs, ECPs, and/or substrates. In addition, polymeric materials are widely used for coatings as paints, in fabrics for cloth etc. Understanding a behavior of these materials in terms of their viscosity enables to produce the electronic devices with a high quality and/or low price. Therefore, viscosity kinetics of the polymeric materials are studied during a material qualification phase, e.g., in order to check whether a production process or a step of the production process is eligible processing the corresponding polymeric material. For example, it may be checked in advance, which viscosity a given polymeric material may have after a certain duration and/or at a given temperature.

However, no method is known to estimate the viscosity of an epoxy resin during its whole curing process.

### DESCRIPTION OF THE INVENTION

It is an objective of the present invention to overcome at least some of the above-mentioned problems.

This objective is achieved by the subject-matter of the independent claim. Further exemplary embodiments are evident from the dependent claims and the following description.

An aspect of the invention relates to a method for estimating a viscosity curve of a polymeric material, the method comprising: acquiring viscosity values of the polymeric material at different temperatures under different heating rates; determining at least one viscosity curve of the polymeric material depending on the measured viscosities for each heating rate; splitting the determined viscosity curve into at least one determined melting curve and at least one determined curing curve per determined viscosity curve; determining at least one fitted melting curve per determined melting curve; determining at least one fitted curing curve per determined curing curve; and estimating the viscosity curve of the polymeric material by combining the fitted melting curve and the fitted curing curve.

The polymeric material may comprise one or more polymers, or a mixture of one or more polymers and one or more fillers. The polymeric material may comprise any polymerizable material, e.g. monomers and/or oligomers.

The splitting of the determined viscosity curve into the determined melting curve and the determined curing curve and determining the corresponding fitted curves separately may contribute to a precise estimation of the viscosity of the polymeric material at the respective temperatures and heating rates. In other words, the estimation of the viscosity, e.g. the viscosity curve, of the polymeric material resulting from the specific temperature and the specific heating rate may be closer to the real viscosity thanks to the splitting of the determined viscosity curve.

According to an embodiment, the fitted curing curve is determined by estimating a curing degree curve for the corresponding heating rate and by determining the fitted curing curve depending on the estimated curing degree curve.

The curing degree curve may contribute to the precise determination of the viscosity of the polymeric material.

According to an embodiment, the curing degree curve is estimated by determining the curing degree of the polymeric material (for example carrying out a DSC analysis on the polymeric material) and by determining at least one linear dependency between the heating rate and the reciprocal temperature (for example from the DSC analysis). The linear dependency may be given by a straight line, wherein the straight line may be referred to as Qi-curve. The linear dependency may be described by a diagram comprising the straight line, wherein the diagram may show a natural logarithm of the heating rate depending on the reciprocal temperature. DSC is a commonly used method for the curing degree determination, which can be carried out easily and accurately. However, another method may be carried out for estimating the curing degree, such as e.g. DEA (Dielectric Analyzer), FTIR (Fourier Transform Infrared Spectroscopy), DMA (Dynamic Mechanics Analyzer), etc.

According to an embodiment, the linear dependency is determined by estimating a fixed value, which is independent from the curing degree; and the linear dependency is determined such that the corresponding straight line comprises the fixed value. The fixed value may be referred to as Qi-point. The use of the Qi-Point in the viscosity estimation may allow a precise estimation of the viscosity curve at the respective temperature and heating rate. In other words, the estimation of the viscosity curve of the polymeric material resulting from the specific temperature and the specific heating rate may be closer to the real viscosity thanks to the involvement of the Qi-Point, as disclosed below, e.g. with respect to the preferred embodiments.

According to an embodiment, the determined viscosity curve is split into the determined melting curve and the determined curing curve at a minimum of the corresponding determined viscosity curve. The minimum viscosity is a precise value for each determined viscosity curve. Therefore, the minimum viscosity is an accurate value for splitting the determined viscosity curve into the determined melting curve and the determined curing curve.

According to an embodiment, the determined viscosity curve is split into at least two determined melting curves; at least one fitted melting curve is determined per split melting curve; and determining a master melting curve by combining the split melting curves; and estimating the viscosity curve of the polymeric material depending on the master melting curve. For example, the determined viscosity curve is split into the determined curing curve and a first determined melting curve and a second determined melting curve. The first determined melting curve may be referred to as α-melting and the second determined melting curve may be referred to as β-melting. Providing two determined melting curves and determining the master melting curve based on the two determined melting curves may contribute to the precise determination of the viscosity curve.

According to an embodiment, the master melting curve is determined by determining an averaged melting curve for the determined melting curves. This may contribute to a precise and/or simple determination of the master melting curve.

According to an embodiment, the master melting curve is fitted based on the Eyring's equation; and the viscosity curve of the polymeric material is determined depending on the fitted master melting curve. The fitting of the master melting curve, in particular based on the Eyring's equation may contribute to the precise determination of the viscosity curve.

According to an embodiment, a maximum viscosity of the polymeric material is determined from a plateau of one of the corresponding determined curing curves. This may contribute to the precise determination of the viscosity curve.

According to an embodiment, the determined curing curve, from which the maximum viscosity is taken, is the determined curing curve with the lowest heating rate. This may contribute to the precise determination of the viscosity curve.

According to an embodiment, a fitting line of the heating rate where the curing degree is 0% corresponds to the temperature with minimum viscosity. This may contribute to the precise determination of the viscosity curve.

According to an embodiment, the fitting line of the heating rate is determined by determining onset temperatures under each heating rate and by adding the determined onset temperatures to the straight lines representing the linear dependencies. This may contribute to the precise determination of the viscosity curve.

According to an embodiment, a temperature at a minimum viscosity is determined depending on a given heating rate; the minimum viscosity is taken from the measured viscosities at different heating rates; and a minimal temperature is fitted to the minimal viscosities by the Eyring's equation. This may contribute to the precise determination of the viscosity curve.

According to an embodiment, the Eyring's equation is applied for determining two or more fitted melting curves per determined melting curve. This may contribute to the precise determination of the viscosity curve.

These and other aspects of the invention will be apparent from and elucidated with reference to the embodiments described hereinafter.

### BRIEF DESCRIPTION OF THE DRAWINGS

Below, embodiments of the present invention are described in more detail with reference to the attached drawings.

FIG. 1 shows a flowchart of an exemplary embodiment of a method for estimating a viscosity curve of a polymeric material;

FIG. 2 shows an example of acquired viscosity values of the polymeric material at different temperatures under different heating rates;

FIG. 3 shows examples of several melting curves of the polymeric material under different heating rates;

FIG. 4 shows examples of several curing curves of the polymeric material under different heating rates;

FIG. 5 shows a flowchart of an exemplary embodiment of a method to estimate a QI-point for a given polymeric material;

FIG. 6 shows an example of a DSC Thermogram of the polymeric material under different heating rates;

FIG. 7 shows an example of a diagram showing curing degrees of the polymeric material depending on the temperature at different heating rates under non-isothermal conditions;

FIG. 8 shows an example of an Ozawa-Flynn-Wall diagram;

FIG. 9 shows an example of a distribution of several intersections;

FIG. 10 shows a detailed view of intersections between 10% and 90% according to FIG. 9;

FIG. 11 shows examples of Qi-curves in a Qi-curve diagram;

FIG. 12 shows a detailed view of the QI-curves according to FIG. 11;

FIG. 13 shows a flowchart of an exemplary embodiment of a method for determining a graph for estimating a conversion degree value of a conversion degree of a polymeric material under non-isothermal conditions;

FIG. 14 shows a diagram including an exemplary graph of conversion degree values depending on the temperature under non-isothermal conditions;

FIG. 15 shows the examples of the several melting curves of the polymeric material and an averaged melting curve;

FIG. 16 shows an example of a master melting curve of the polymeric material fitted by the averaged melting curve;

FIG. 17 shows an example of a fitting curve for fitting a minimum viscosity with regard to a minimum temperature.

FIG. 18 shows a detailed flowchart of an exemplary embodiment of a method for estimating a viscosity curve of a polymeric material;

The reference symbols used in the drawings, and their meanings, are listed in summary form in the list of reference symbols. In principle, identical parts are provided with the same reference symbols in the figures.

### DETAILED DESCRIPTION OF EXEMPLARY EMBODIMENTS

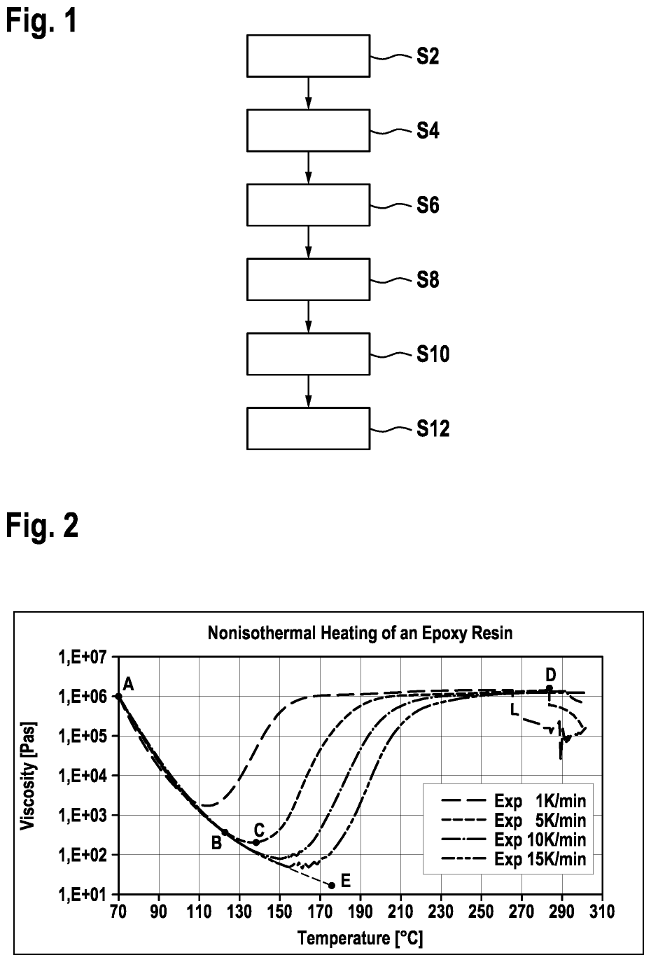

FIG. 1 shows a flowchart of an exemplary embodiment of a method for estimating a viscosity curve of a polymeric material. The polymeric material may comprise one or more polymers, or a mixture of one or more polymers and one or more fillers. The polymeric material may comprise any polymerizable material, e.g. monomers and/or oligomers.

In a step S2, viscosity values of the polymeric material are acquired at different temperatures T under different heating rates β. The viscosity values may acquired by heating up the polymeric material under the different heating rates and by measuring the viscosity of the polymeric material at the different temperatures T.

In a step S4, at least one viscosity curve of the polymeric material is determined depending on the measured viscosities for each heating rate β. One example of a corresponding viscosity curve is shown in FIG. 2. The viscosity curve may be determined by connecting the measured viscosity values in a corresponding diagram, e.g. as shown in FIG. 2. The viscosity value at a beginning of the measurement may correspond to a first characteristic point A shown in FIG. 2.

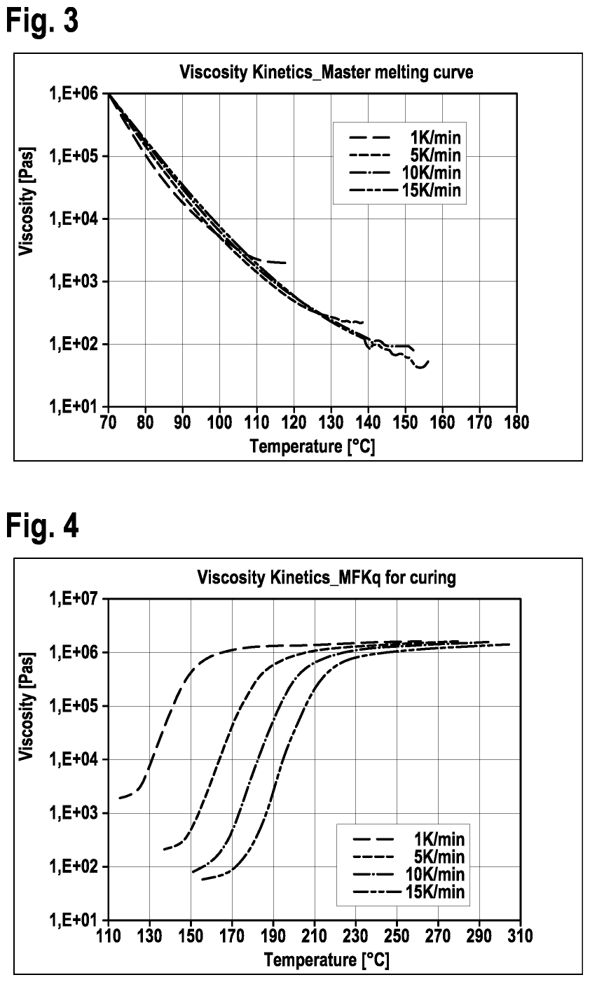

In a step S6, the determined viscosity curve is split into at least one determined melting curve and at least one determined curing curve per determined viscosity curve. Several examples of corresponding melting curves are shown in FIG. 3. Several examples of corresponding curing curves are shown in FIG. 4. Optionally, as explained later, the determined viscosity curve may be split into two or more determined melting curves and one determined curing curve. For example, with respect to two melting curves, a first melting curve may extend from the first characteristic point A to a second characteristic point B of the determined viscosity curve, and a second melting curve may extend from the second characteristic point B to a third characteristic point C of the determined viscosity curve.

Optionally, the determined viscosity curve may be split into the determined melting curve and the determined curing curve at a minimum of the corresponding determined viscosity curve, wherein the minimum is shown in FIG. 2 as the third characterizing point C. The minimum viscosity ηₘᵢₙ is a precise value for each determined viscosity curve. A temperature at the minimum viscosity ηₘᵢₙ may be determined depending on a given heating rate β. The minimum viscosity ηₘᵢₙ may be taken from the measured viscosities at different heating rates β. A minimal temperature Tₘᵢₙ may be fitted to the minimal viscosities ηₘᵢₙ by the Eyring's equation:

$$η = \frac{{hN}_{A}}{{V}_{m}} {e}^{\frac{{E}_{0}}{RT}}$$

with η being the dynamic viscosity, h being the Planck's constant, N_{A} being the Avogadro's number, Vₘ being the molar volume of the polymeric material, E₀ being an activation free energy for viscous flow, and R being the gas constant.

Optionally, the Eyring's equation may be applied for determining two or more fitted melting curves per determined melting curve. In particular, the determined viscosity curve may be split into at least two determined melting curves. At least one fitted melting curve may be determined per split melting curve. Then, a master melting curve may be determined by combining the split melting curves. The viscosity curve of the polymeric material may be determined depending on the master melting curve. For example, the determined viscosity curve is split into the determined curing curve and a first determined melting curve and a second determined melting curve. The first determined melting curve may be referred to as α-melting, e.g. between the characterizing points A and B, and the second determined melting curve may be referred to as β-melting, e.g. between the characterizing points B and C.

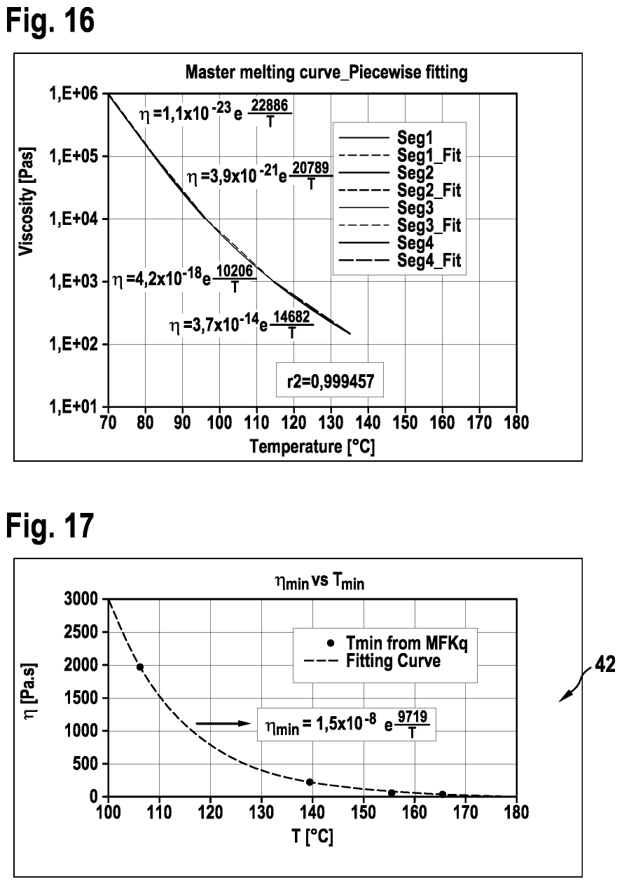

The master melting curve may be determined by determining an averaged melting curve for the determined melting curves. One example of a corresponding averaged melting curve is shown in FIG. 15. Then, the master melting curve may be fitted based on the Eyring's equation, e.g. as shown in FIG. 16. Then, the viscosity curve of the polymeric material may be estimated depending on the fitted master melting curve.

Optionally, a maximum viscosity ηₘₐₓ of the polymeric material may be determined from a plateau of one of the corresponding determined curing curves. For example, the determined curing curve, from which the maximum viscosity ηₘₐₓ is taken, may be the determined curing curve with the lowest heating rate β.

In a step S8, at least one fitted melting curve is determined per determined melting curve. Several examples of correspondingly fitted melting curves are shown in FIG. 16.

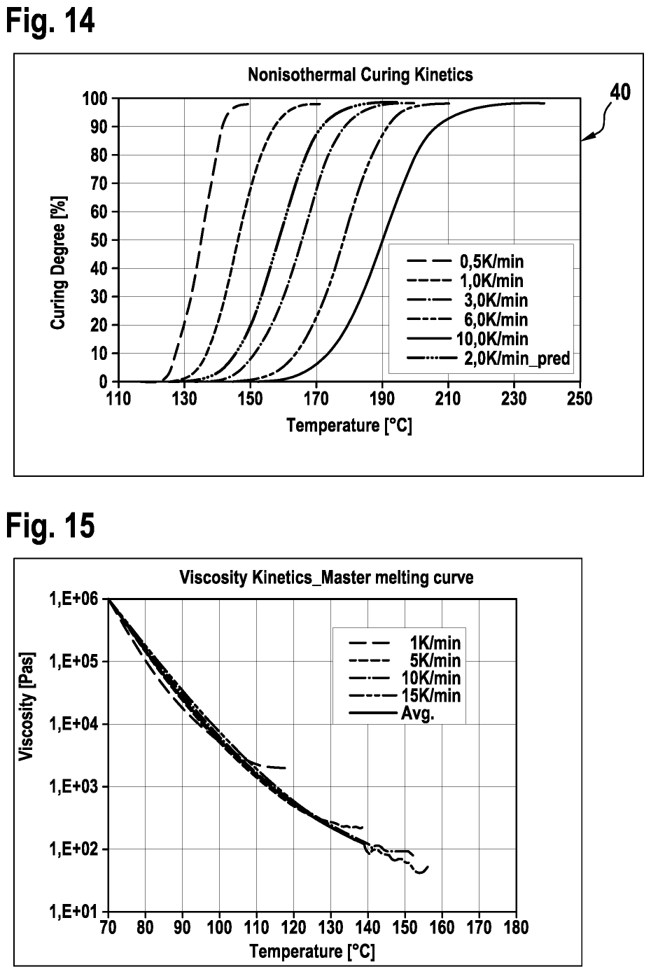

In a step S10, at least one fitted curing curve is determined per determined curing curve. Several examples of correspondingly fitted curing curves are shown in FIG. 14. The fitted curing curve may be determined by estimating a curing degree curve for the corresponding heating rate β and by determining the fitted curing curve depending on the estimated curing degree curve. The curing degree curve may contribute to the precise determination of the viscosity of the polymeric material. The curing degree curve may be determined by the method explained with respect to FIG. 13.

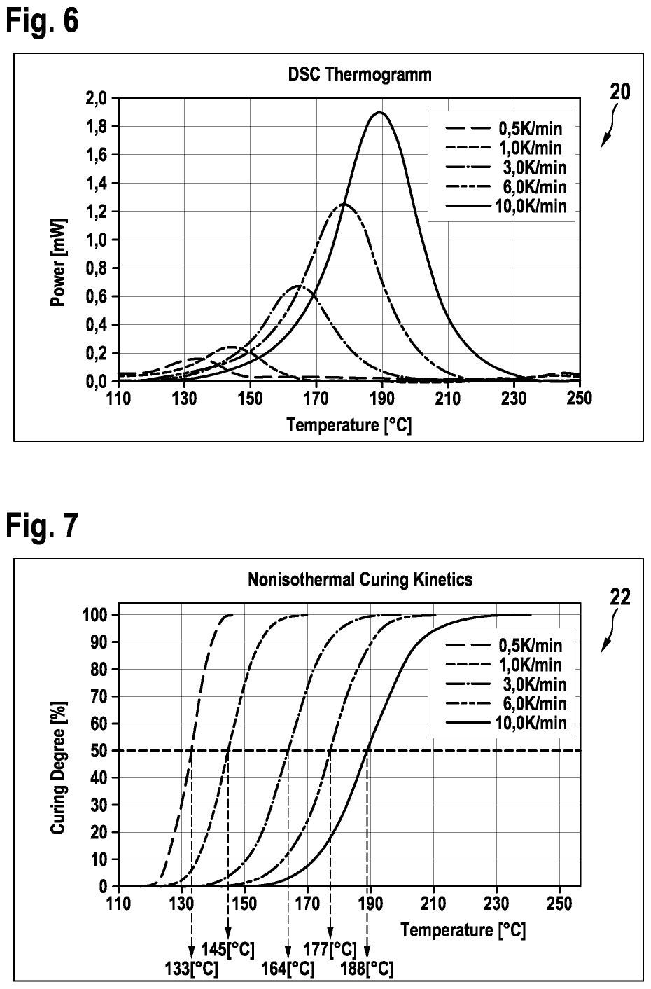

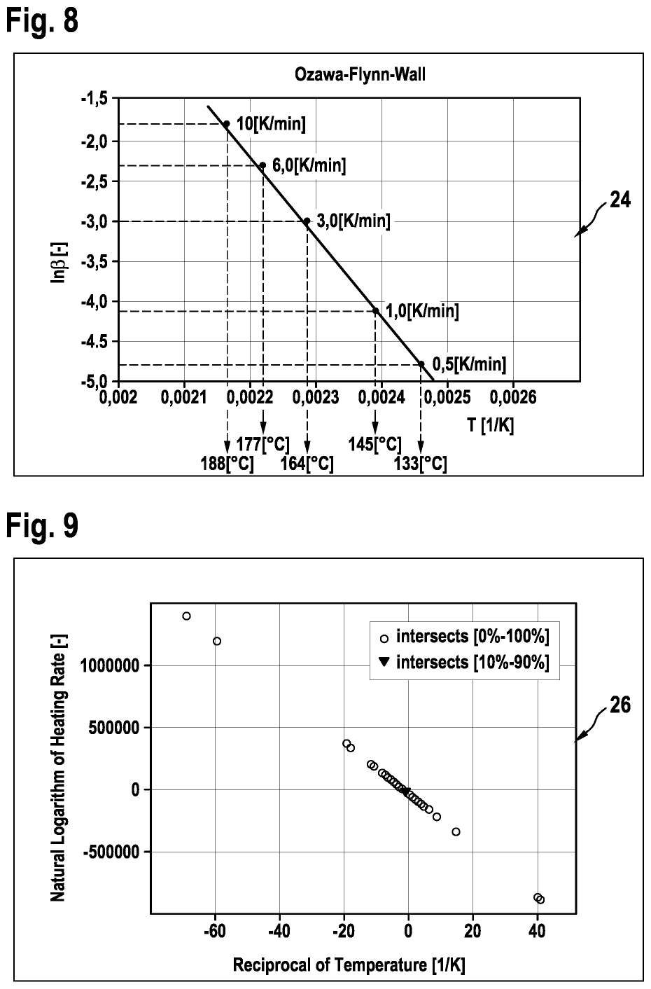

In particular, the curing degree curve may be estimated by carrying out a DSC analysis (see FIG. 6) on the polymeric material and by determining at least one linear dependency (FIGS. 8 and 11) between the heating rate β and the reciprocal temperature 1/T from the DSC analysis. The linear dependency may be given by a straight line, wherein the straight line may be referred to as Qi-curve. The linear dependency may be described by a diagram comprising the straight line, wherein the diagram may show a natural logarithm of the heating rate β depending on the reciprocal temperature 1/T.

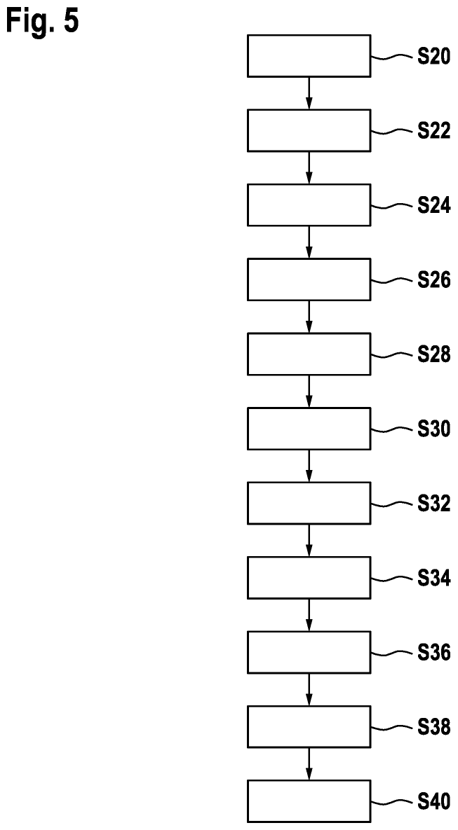

The linear dependency may be determined by estimating a fixed value, which is independent from the curing degree, and the linear dependency may be determined such that the corresponding straight line comprises the fixed value. The fixed value may be referred to as Qi-point. The Qi-point may be determined by the method explained with respect to FIG. 5.

Optionally, a fitting line of the heating rate β where the curing degree is 0% may correspond to the temperature T with minimum viscosity ηₘᵢₙ. Alternatively or additionally, the fitting line of the heating rate β may be determined by determining onset temperatures Tₒₙₛₑₜ under each heating rate β and by adding the determined onset temperatures Tₒₙₛₑₜ to the straight lines representing the linear dependencies.

In a step S12, the viscosity curve of the polymeric material is estimated by combining the fitted melting curve(s) and the corresponding fitted curing curve(s).

FIG. 2 shows examples of acquired viscosity values η of the polymeric material at different temperatures T under different heating rates β, in particular as examples of the determined viscosity curves. When carrying out the method explained with respect to FIG. 1, the corresponding viscosity curves may be determined, wherein the corresponding viscosity values n may be acquired at different heating rates β such that one viscosity curve may be determined per heating rate β.

An onset of the β-melting of the viscosity curve corresponding to the heating rate β°=°5° K/min is given at the characterizing point B. The onset temperature Tₒₙₛₑₜ and the onset viscosity ηₒₙₛₑₜ are the temperature and, respectively, the viscosity at the characterizing point B and are to be defined by the method explained with respect to claim 1.

The curing starts at the characterizing point C of the viscosity curve corresponding to the heating rate β°=°5° K/min. The minimum temperature Tₘᵢₙ and the minimum viscosity ηₘᵢₙ are the temperature and, respectively, the viscosity at the characterizing point C and are to be defined by the method explained with respect to claim 1.

The maximum viscosity ηₘₐₓ before a decomposition starts is given at the characterizing point D of the viscosity curve corresponding to the heating rate β°=°5° K/min. The maximum viscosity ηₘₐₓ depends on the heating rate β, wherein a theoretical maximum viscosity of the polymeric material may not be achieved yet (in the diagram shown in FIG. 2).

FIG. 3 shows examples of several determined melting curves of the polymeric material. The determined melting curves represent the α-melting, in particular between the corresponding characterizing points A and B of the corresponding viscosity curve. The melting curves shown in FIG. 3 are determined by the above described measurement and by splitting the corresponding determined viscosity curves.

FIG. 4 shows examples of several determined curing curves of the polymeric material. The determined curing curves may extend from the corresponding characterizing points C to corresponding fourth characterizing points D (see FIG. 2). From FIG. 4, the minimal viscosities ηₘᵢₙ may be extracted, e.g.

$${η}_{min} = 1 972.8 Pa \cdot s for β = 1 K / min$$

$${η}_{min} = 255. Pa \cdot s for β = 5 K / min$$

$${η}_{min} = 89.6 Pa \cdot s for β = 10 K / min$$

$${η}_{min} = 61.5 Pa \cdot s for β = 15 K / min$$

FIG. 5 shows a flowchart of an exemplary embodiment of a method to estimate the Qi-point QI (see FIG. 11) for the polymeric material.

In a step S20, the Differential Scanning calorimetry (DSC) measurement of a probe of the polymeric material may be carried out under n different heating rates βₙ°=°[β₀, β₁, . . . , βₙ₋₁], with n being a natural number. For example, n may be in the range from 1 to 20, e.g. from 1 to 10, e.g. from 3 to 6.

FIG. 6 shows an example of a DSC thermogram 20 of the polymeric material determined by the DSC measurement. The DSC thermogram 20 may comprise one graph per heating rate β, wherein the heating rates βₙ are given in K/min.

Alternatively, the heating rate β may be given in any other possible temperature to time relation, e.g. ° C./s or ° F./h.

A curing degree value α of a curing degree may be determined from the DSC thermogram 20, wherein the curing degree value α may be determined by the formula:

$$∝ = \frac{{H}_{T}}{{H}_{T o t a l}} \times 100 \%$$

with H_{Total} being the energy which is absorbed by the polymeric material until the curing of the polymeric material is finished and with H_{Total} corresponding to the area under the corresponding graph of FIG. 2; and with H_{T} being the energy which is absorbed by the polymeric material until the temperature T is reached and with H_{T} corresponding to the area under the corresponding graph of FIG. 2 from the very left to the Temperature T.

In a step S22, the curing degree values α°=°[0, 0.01, . . . , 100] (in sum 10001 elements) and the corresponding temperatures Tᵢ=[T_{i,0}, T_{i,1}, . . . , T_{i,1000}] will be determined for each heating rate βᵢ, with i°=°0, °1, ° . . . , °m−1 being a natural number.

FIG. 7 shows an example of a diagram, which may be referred to as first diagram 22 in the following. The first diagram 22 shows the determined curing degree values α of the polymeric material depending on the temperatures Tᵢ at the different heating rates βₙ under non-isothermal conditions. From FIG. 3 it may be seen that the curing degree is a function of the heating rate β and the temperature T.

∝=∝(β,T)

For example, the dashed horizontal line within the first diagram 22 may correspond to a curing degree value α of 50%, i.e. α°=°50°%, wherein the intersections of the graphs of the curing degrees α with that dashed horizontal line provide the temperatures T at which the curing degree value α is 50% under the corresponding heating rate β.

FIG. 8 shows an example of an Ozawa-Flynn-Wall diagram, which may be referred to as second diagram 24. The second diagram 24 may be achieved by an Ozawa-Flynn-Wall Analysis, as it is known in the art. The Ozawa-Flynn-Wall diagram of FIG. 4 may be constructed for the curing degree α°=°50°% by the heating rates βₙ and temperatures T extracted from the diagram of FIG. 3 by the corresponding horizontal line, as explained above.

The Ozawa-Flynn-Wall diagram may also comprise graphs correspondingly constructed for the other curing degrees α°=°[0, 0.01, . . . , 100]. However, the corresponding graphs constructed according to the conventional Ozawa-Flynn-Wall Analysis may intersect each other in a region of the Ozawa-Flynn-Wall diagram (not shown in the figures), what makes no sense from a physical point of view and what shows the drawbacks of the conventional Ozawa-Flynn-Wall Analysis. Therefore, instead of using the conventional Ozawa-Flynn-Wall Analysis, the present inventor found a more accurate way of constructing the graphs for predicting the curing degree α for the given polymeric material, as explained in the following.

In a step S24, the graphs in the Ozawa-Flynn-Wall diagram are plotted for all of the above Temperatures Tᵢ, heating rates βₙ, and curing degrees α°=°[0, ° 0.01, ° . . . , °100] according to

$$[ ( \frac{1}{{T}_{0 , j}} , ln {β}_{0} ) , ( \frac{1}{{T}_{1 , j}} , ln {β}_{1} ) , … , ( \frac{1}{{T}_{m - 1 , j}} , ln {β}_{n - 1} ) ]$$

with j°=°0, 1, . . . , 10000, for example. So, the corresponding diagram, which may be referred to as Qi-curve diagram, may e.g. comprise 10001 graphs.

In a step S26, the graphs of the Qi-curve diagram may be linearly fitted and the slopes and intercepts with the y-axis of the correspondingly fitted graphs, i.e. Qi-curves, may be extracted, resulting in slopes K°=°[k₀, k₁, . . . , k₁₀₀₀₀] and intercepts B°=°[b₀, b₁, . . . , b₁₀₀₀₀].

In a step S28, the intersections of the fitted graph for the curing degree value α between 1% and 30%, e.g. between 5% and 15%, e.g. α°=°10°%, with all other fitted graphs may be determined, e.g. by

$$\{ \begin{matrix}ln β = {k}_{1000} \frac{1}{T} + {b}_{1000} \\ ln β = {k}_{i} \frac{1}{T} + {b}_{i} , ( i = 0 , … , 10000 , i \neq 1000 )\end{matrix}$$

$$[ ( \frac{1}{{T}_{0}} , ln {β}_{0} ) , ( \frac{1}{{T}_{1}} , ln {β}_{1} ) , … , ( \frac{1}{{T}_{9999}} , ln {β}_{9999} ) ]$$

In a step S30, the intersections of the fitted graph for the curing degree value α between 30% and 60%, e.g. between 40% and 55%, e.g. α°=°50°%, with all other fitted graphs may be determined, e.g. by

$$\{ \begin{matrix}ln β = {k}_{5000} \frac{1}{T} + {b}_{5000} \\ ln β = {k}_{i} \frac{1}{T} + {b}_{i} , ( i = 0 , … , 10000 , i \neq 5000 )\end{matrix}$$

$$[ ( \frac{1}{{T}_{10000}} , ln {β}_{10000} ) , … , ( \frac{1}{{T}_{19999}} , ln {β}_{19999} ) ]$$

In a step S32, the intersections of the fitted graph for the curing degree value α between 70% and 99%, e.g. between 85% and 95%, e.g. α°=°90°%, with all other fitted graphs may be determined, e.g. by

$$\{ \begin{matrix}ln β = {k}_{9000} \frac{1}{T} + {b}_{9000} \\ ln β = {k}_{i} \frac{1}{T} + {b}_{i} , ( i = 0 , … , 10000 , i \neq 9000 )\end{matrix}$$

$$[ ( \frac{1}{{T}_{20000}} , ln {β}_{20000} ) , … , ( \frac{1}{{T}_{29999}} , ln {β}_{29999} ) ]$$

In a step S34, the intersections determined in the steps S10 to S14 may be plotted in a third diagram 26, e.g. as shown in FIG. 9.

FIG. 9 shows an example of a distribution of several of the above intersections. The distribution of the intersections is shown in the third diagram 26. From FIG. 9 it may be seen that 80% of the intersections lie around the reciprocal temperature 0, wherein the reciprocal temperature of 0 may only be achieved for the temperature T going towards infinite.

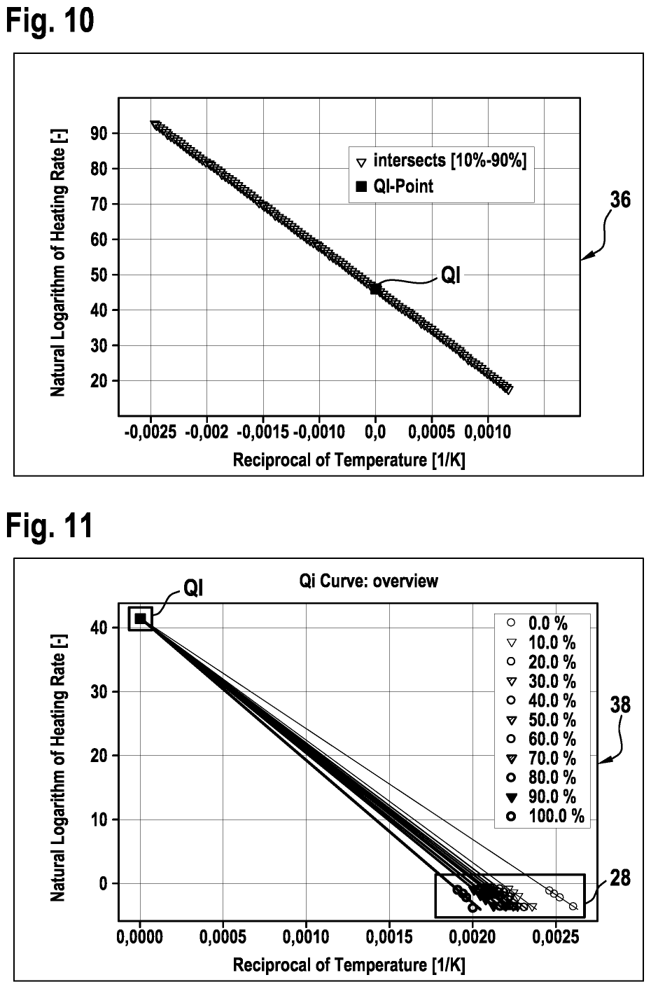

FIG. 10 shows a detailed view of the intersections between 10% and 90% in a fourth diagram 36.

In a step S36, the intersection of the fitted graph with 1/T°=°0 may be determined as the Qi-point QI, with QI°=°(0, ln β).

FIG. 11 shows examples of Qi-curves, all of which including the Qi-point QI, in a fifth diagram 38. The Qi-curves do not intersect each other except for the Qi-point QI, what perfectly makes sense from a physical point of view.

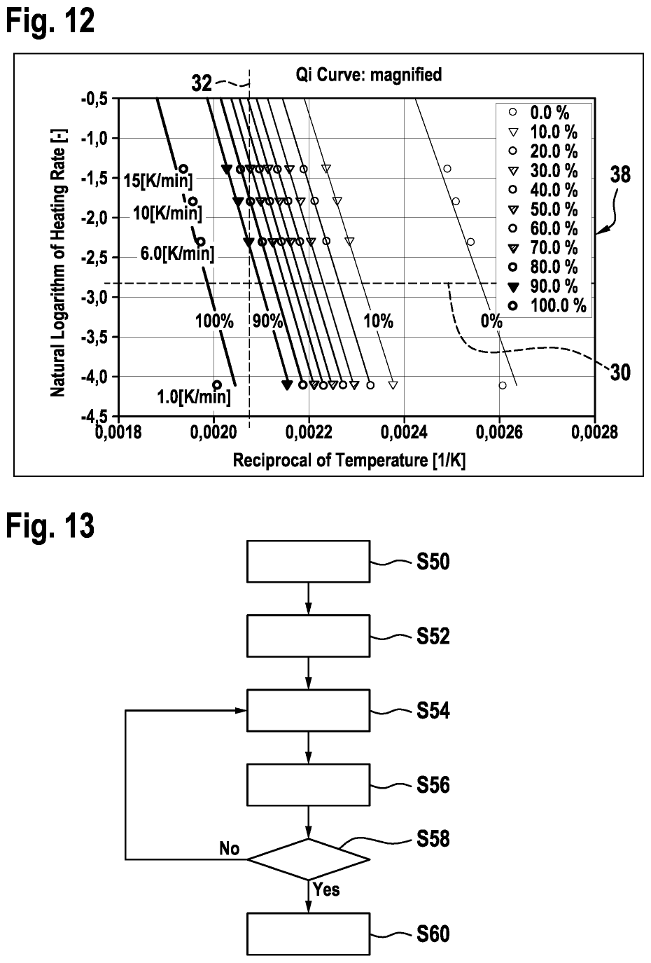

FIG. 12 shows a detailed view of the QI-curves according to FIG. 11, in particular a view of a lower area 28 of the fifth diagram 38.

The Qi-curves of FIGS. 11 and 12 may be determined by steps S38 and S40.

In the step S38, the graphs, i.e. the Qi-curves, are plotted again according to the Ozawa-Flynn-Wall equation

$$ln ( β ) = ( - \frac{{E}_{α}}{R} ) \frac{1}{T} + \{ ln [ Af ( α ) ] - ln ( \frac{d α}{d T} ) \}$$

and by using the Qi-point QI, wherein the term (−E_{α}/R) defines the slope, with R being the ideal gas constant, and the term {ln[Af(α)]−ln(dα/dT)} defines the intercept of the corresponding curves, with A being the pre-exponential factor with the unit [1/s].

$$[ ( \frac{1}{{T}_{0 , j}} , ln {β}_{0} ) , ( \frac{1}{{T}_{1 , j}} , ln {β}_{1} ) , … , ( \frac{1}{{T}_{m - 1 , j}} , ln {β}_{n - 1} ) , ( 0 , ln {β}_{q} ) ]$$

with j°=°0, 1, . . . , 10000, for example.

In step S40, the graphs may be linearly fitted again in the Qi-diagram in order to obtain the Qi-curves, which correspond to the Ozawa-Flynn-Wall curves including the Qi-point QI, and correspondingly adapted slopes K′=°[k′₀, °k′₁, ° . . . , °k′₁₀₀₀₀] and intercepts B′=°[b′₀, b′₁, . . . , b′₁₀₀₀₀] may be extracted.

FIG. 13 shows a flowchart of an exemplary embodiment of a method for determining a graph for estimating a conversion degree value of a conversion degree of a polymeric material under non-isothermal conditions. The conversion degree may be the curing degree. The method may use the above predetermined curing degree values αᵢ, heating rates β and determined Qi-point QI and may determine the corresponding temperatures T at which the predetermined curing degree values αᵢ are reached in order to estimate a continuous progression and/or behaviour of the curing degree values αᵢ in form of a mathematical function and/or a corresponding graph depending on the temperature T such that one or more curing degree values α at one or more desired and/or arbitrary temperatures T may be extracted by the mathematical function and/or from the corresponding graph afterwards.

In a step S50, a given heating rate β is received for which the graph representing the curing degree values α of the curing degree of the polymeric material depending on the temperature T shall be estimated. The heating rate β may be input into the device for determining the graph for estimating the curing degree value α and the device may receive the heating rate β.

In a step S52, the index i and the curing degree value αᵢ each are set to 0, and the above curing degree values αᵢ°=°[0, 0.01, . . . , 100] and the above Qi-point QI°=°(0, ln β) are received by the device. Further, the slope of the graph may be given as the above function f(α).

In a step S54, Tᵢ may be determined by

$${T}_{i} = \frac{f ( {α}_{i} )}{ln ( β ) - ln ( {β}_{q} )}$$

In a step S56, the index i is incremented by 1, i.e. i°=°i°+°1, and the next conversion degree value αᵢ is chosen, as e.g. by αᵢ°=αᵢ₋₁°+°δα, wherein δα may for example be 1.

In a step S58, it is checked whether the current conversion degree value ai is larger than 100. If the condition of step S58 is not fulfilled, the method may proceed in step S54. If the condition of step S58 is fulfilled, the method may proceed in step S60.

In step S60, the graph representing the curing degree α over the temperature T may be plotted, e.g. as shown in FIG. 14, and/or the method for determining the graph for estimating the conversion degree values α of the conversion degree of the polymeric material under non-isothermal conditions may be terminated.

The above method for determining the graph for estimating a conversion degree value α of the conversion degree of the polymeric material under non-isothermal conditions may be used as a sub-routine of the method for estimating the viscosity curve of the polymeric material under non-isothermal conditions, as explained with respect to FIG. 1.

FIG. 14 shows a diagram including exemplary graphs of the conversion degree α depending on the temperature T under non-isothermal conditions, in particular for different given heating rates β. The diagram may be referred to as sixth diagram 40. The sixth diagram 40 may be determined by the above method for determining the graph for estimating the conversion degree value α of the conversion degree of the polymeric material under non-isothermal conditions.

FIG. 15 shows the examples of the several determined melting curves of the polymeric material and an averaged melting curve. In particular, FIG. 15 may correspond to FIG. 3 except for the averaged melting curve. The averaged melting curve may be determined by any known averaging method based on the several determined melting curves. The averaged melting curve may be provided for a piecewise fitting procedure.

FIG. 16 shows an example of a master melting curve of the polymeric material fitted by the averaged melting curve. The master melting curve may be determined from the averaged melting curve by the piecewise fitting based on the Eyrings's equation, as it is known in the art.

FIG. 17 shows an example of a fitting curve for fitting the minimum viscosity ηₘᵢₙ with regard to the minimum temperature Tₘᵢₙ. The fitting curve may be referred to as seventh diagram 42.

In particular, for a given heating rate β the temperature T at the minimum viscosity ηₘᵢₙ is determined based on the relationship given by the Qi-curves. Then, the minimum viscosity ηₘᵢₙ from the above measurement is taken, i.e. the minimum viscosity ηₘᵢₙ at the characterizing point C. Afterwards, the temperature at the minimum viscosity ηₘᵢₙ is fitted by means of the Eyring's equation.

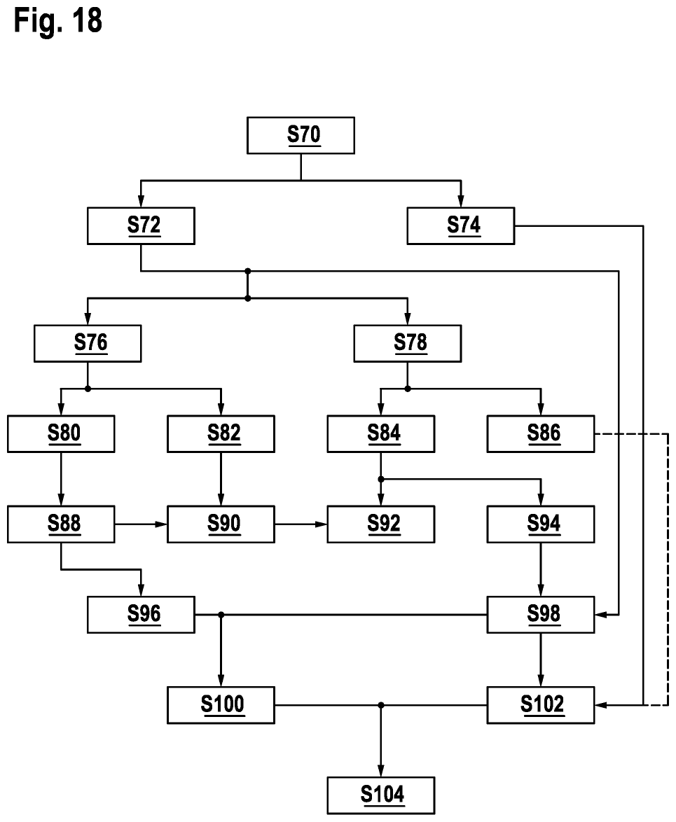

FIG. 18 shows a detailed flowchart of an exemplary embodiment of a method for estimating a viscosity curve of a polymeric material. In particular, with respect to FIG. 18 a very sophisticated method for estimating the viscosity curve is explained, wherein the more general method explained with respect to FIG. 1 may comprise the specific method of FIG. 18. So, the above diagrams and calculations are also valid for the method of FIG. 18, wherein repetitions are omitted in order to provide a concise description of the invention.

In a step S70, the viscosity curves depending on the temperature T under different heating rates β may be determined.

In a step S72, the minimum viscosities ηₘᵢₙ at different heating rates β may be determined from the determined viscosity curves.

In a step S74, the maximum viscosity ηₘₐₓ may be determined for all heating rates β.

In a step S76, the determined melting curves may be extracted from the determined viscosity curves.

In a step S78, the determined curing curves may be extracted from the determined viscosity curves.

In a step S80, the α-melting curves may be determined from the determined melting curves.

In a step S82, the β-melting curves may be determined from the determined melting curves.

In a step S84, the Qi-curves may be determined from the determined curing curves.

In a step S86, the curing degree values α depending on the heating rate β and the temperature T may be determined.

In a step S88, the viscosity η for the α-melting curves may be determined from the α-melting curves and by use of the Eyring's equation as

$${η}_{α - m e l t i n g} = {a}_{0} {e}^{\frac{{b}_{0}}{{T}_{o n s e t}}}$$

In a step S90, the onset temperatures Tₒₙₛₑₜ at different heating rates β may be determined from the viscosity η for the α-melting curves and from the β-melting curves.

In a step S92, the heating rates β at the onset temperatures Tₒₙₛₑₜ may be determined from the onset temperatures Tₒₙₛₑₜ at different heating rates β and from the Qi-curves.

In a step S94, the heating rates β at the minimal temperature Tₘᵢₙ (which is the temperature at the minimum viscosity ηₘᵢₙ) may be determined from the Qi-curves.

In a step S96, the onset viscosity ηₒₙₛₑₜ may be determined from the viscosity η for the α-melting curves and depending on the heating rate β at the onset temperature Tₒₙₛₑₜ as

$${η}_{o n s e t} = {a}_{0} {e}^{\frac{{b}_{0}}{{T}_{o n s e t}}}$$

In a step S98, the minimum viscosity ηₘᵢₙ may be determined from the determined minimum viscosities ηₘᵢₙ at different heating rates β and depending on the heating rate β at the minimum temperature Tₘᵢₙ with the help of the Eyring's equation as

$${η}_{min} = {a}_{0} {e}^{\frac{{b}_{2}}{{T}_{min}}}$$

In a step S100, the viscosity η for the β-melting curves may be estimated from the onset viscosity ηₒₙₛₑₜ and the minimum viscosity ηₘᵢₙ, and by use of the Eyring's equation as

$${η}_{β - melting} = {a}_{1} {e}^{\frac{{b}_{1}}{{T}_{o n s e t}}}$$

In a step S102, the viscosity η for the curing curves may be estimated from the minimum viscosity ηₘᵢₙ, the minimum viscosity ηₘᵢₙ at different heating rates β, and the maximum viscosity ηₘₐₓ for all heating rates β as

$${η}_{c u r i n g} = α ( β , T ) \cdot ( {η}_{max} - {η}_{min} ) + {η}_{min}$$

In a step S104, the estimated melting curve and the estimated curing curve are joined to the estimated viscosity curve. In other words, the estimated viscosity curve may be provided by the estimated melting curve and the estimated curing curve.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practising the claimed invention, from a study of the drawings, the disclosure, and the appended claims. In the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

LIST OF REFERENCE SYMBOLS

| | |
|---|---|
| 20 | DSC Thermograph |
| 22 | first diagram |
| 24 | second diagram |
| 26 | third diagram |
| 28 | lower area |
| 30 | isothermal line |
| 32 | non-isothermal line |
| 36 | fourth diagram |
| 38 | fifth diagram |
| 40 | sixth diagram |
| 42 | seventh diagram |
| QI | Qi-point |
| A-C | characteristic points |
| S2-S104 | steps two to one hundred and four |

1. A method for estimating a viscosity curve of a polymeric material, the method comprising:
acquiring viscosity values of the polymeric material at different temperatures (T) under different heating rates (β);
determining a viscosity curve of the polymeric material depending on measured viscosities for each heating rate (β);
splitting the viscosity curve into at least one determined melting curve and at least one determined curing curve per determined viscosity curve;
determining at least one fitted melting curve per determined melting curve;
determining at least one fitted curing curve per determined curing curve; and
estimating the viscosity curve of the polymeric material by combining the fitted melting curve and the fitted curing curve.

2. The method in accordance with claim 1, wherein
the fitted curing curve is determined by estimating a curing degree curve for a corresponding heating rate (β) and by determining the fitted curing curve depending on the estimated curing degree curve.

3. The method in accordance with claim 2, wherein
the curing degree curve is estimated by determining the curing degree of the polymeric material and by determining at least one linear dependency between the heating rate (β) and a reciprocal temperature.

4. The method in accordance with claim 3, wherein
the linear dependency is determined by estimating a fixed value, which is independent from the curing degree; and
the linear dependency is determined such that a corresponding straight line comprises the fixed value.

5. The method in accordance with claim 1, wherein
the determined viscosity curve is split into the determined melting curve and the determined curing curve at a minimum (ηₘᵢₙ) of a corresponding determined viscosity curve.

6. The method in accordance with claim 1, wherein
the determined viscosity curve is split into at least two determined melting curves;
at least one fitted melting curve is determined per split melting curve; and
determining a master melting curve by combining the split melting curves; and
estimating the viscosity curve of the polymeric material depending on the master melting curve.

7. The method in accordance with claim 6, wherein
the master melting curve is determined by determining an averaged melting curve for the determined melting curves.

8. The method in accordance with claim 7, wherein
the master melting curve is fitted based on Eyring's equation; and
the viscosity curve of the polymeric material is determined depending on the fitted master melting curve.

9. The method in accordance with claim 1, wherein
a maximum viscosity of the polymeric material is determined from a plateau of one of the at least one determined curing curves.

10. The method in accordance with claim 8, wherein
the determined curing curve, from which a maximum viscosity is taken, is the determined curing curve with a lowest heating rate (β).

11. The method in accordance with claim 1, wherein
a fitting line of the heating rate (β) where a curing degree is 0% corresponds to the temperature with minimum viscosity (ηₘᵢₙ).

12. The method in accordance with claim 11, wherein
the fitting line of the heating rate (β) is determined by determining onset temperatures (Tₒₙₛₑₜ) under each heating rate (β) and by adding the determined onset temperatures (Tₒₙₛₑₜ) to straight lines representing linear dependencies.

13. The method in accordance with claim 8, wherein
a temperature at a minimum viscosity (ηₘᵢₙ) is determined depending on a given heating rate (β);
minimum viscosities (ηₘᵢₙ) are taken from the measured viscosities at different heating rates (β); and
a minimal temperature (Tₘᵢₙ) is fitted to the minimal viscosities (ηₘᵢₙ) by the Eyring's equation.

14. The method in accordance with claim 8, wherein
the Eyring's equation is applied for determining two or more fitted melting curves per determined melting curve.
